(19) United States
(12) Patent Application Publication
(10) Pub. No.: US 2021/0401300 A1
(43) Pub. Date: Dec. 30, 2021

(54) BIOLOGICAL INFORMATION MEASURING DEVICE

(71) Applicant: SONY GROUP CORPORATION, Tokyo (JP)

(72) Inventors: Atsushi ITO, Tokyo (JP); Yoshiro TAKIGUCHI, Tokyo (JP)

(73) Assignee: SONY GROUP CORPORATION, Tokyo (JP)

(21) Appl. No.: 17/290,482

(22) PCT Filed: Sep. 18, 2019
(86) PCT No.: PCT/JP2019/036645

§ 371 Date: Apr. 30, 2021

(30) Foreign Application Priority Data
Nov. 12, 2018 (JP) 2018-212356

(51) Int. Cl. A61B 5/026

(52) U.S. Cl. CPC A61B 5/026; A61B 2562/185

(57) ABSTRACT

To provide a biological information measuring device capable of suppressing positional displacement between a light emitting element and a light receiving element, and an opening formed on a light shielding film. A biological information measuring device provided with a plate-shaped lid having translucency, a first light shielding film provided on a first principal surface of the lid and having conductivity, a light emitting element, and a light receiving element, in which the light emitting element and the light receiving element are electrically joined to the first light shielding film, and the first light shielding film includes a first light emitting side opening through which light emitted from the light emitting element passes, and a first light receiving side opening through which light received by the light receiving element passes.

### TECHNICAL FIELD

The present technology relates to a biological information measuring device.

### BACKGROUND ART

A technology referred to as laser Doppler flowmetry is known that irradiates skin with coherent light and analyzes backscattered light thereof, thereby non-invasively measuring a blood flow speed under the skin, and various blood flow measuring devices using this technology are provided. For example, in a sensor unit for blood flow meter disclosed in Patent Document 1, a cover substrate obtained by forming a light shielding film that shields unnecessary scattered light on a semiconductor substrate on which a light emitting element and a light receiving element are formed is arranged. Patent Document 1 also discloses a configuration in which an opening for allowing scattered light from living body tissue to be incident on the light receiving element is provided on the light shielding film. A package for measurement sensor disclosed in Patent Document 2 is provided with a ground conductor layer on a lid that covers a light emitting element and a light receiving element, and the ground conductor layer serves as a mask member that blocks unnecessary light. The ground conductor layer of Patent Document 2 is provided with a first opening through which light emitted from the light emitting element passes and a second opening through which light received by the light receiving element passes.

In a case where structures disclosed in Patent Documents 1 and 2 are adopted, as a manufacturing procedure, it is common to mount the light emitting element and the light receiving element on a housing or a substrate including metal wiring, and thereafter cover the same with a member on which the light shielding film is formed. For example, the package for measurement sensor disclosed in Patent Document 2 may be manufactured by mounting the light emitting element and the light receiving element on the substrate, and thereafter covering the same with the lid provided with the ground conductor layer on which the first opening and the second opening are formed.

However, in such manufacturing procedure, it is highly possible that, when various parts are combined, displacement occurs in relative positions between the parts, and the displacement is accumulated to finally cause positional displacement between the light emitting element and light receiving element and the openings formed on the light shielding film. In this manner, in the conventional technology, it is difficult to precisely adjust the relative positions of the light emitting element and light receiving element and the openings formed on the light shielding film.

### CITATION LIST

### Patent Document

Patent Document 1: Japanese Patent Application Laid-Open No. 2004-229920
Patent Document 2: Japanese Patent Application Laid-Open No. 2017-131286

### SUMMARY OF THE INVENTION

### Problems to be Solved by the Invention

Therefore, it is a principal object of the present technology to provide a biological information measuring device capable of suppressing positional displacement between a light emitting element and a light receiving element, and openings formed on a light shielding film.

### Solutions to Problems

That is, the present technology provides

a biological information measuring device, provided with:

a plate-shaped lid having translucency; a first light shielding film provided on a first principal surface of the lid and having conductivity; a light emitting element; and a light receiving element,

in which the light emitting element and the light receiving element are electrically joined to the first light shielding film, and

the first light shielding film includes a first light emitting side opening through which light emitted from the light emitting element passes, and a first light receiving side opening through which light received by the light receiving element passes.

The first light shielding film may be divided into three or more sections independent from each other, and at least one of the terminal of the light emitting element or the terminal of the light receiving element may be respectively joined to the sections.

The first light shielding film may include a light reflecting material.

The biological information measuring device may be provided with a second light shielding film provided on a second principal surface facing the first principal surface of the lid, in which the second light shielding film may include a second light receiving side opening through which light received by the light receiving element passes.

The second light shielding film may include a second light emitting side opening through which light emitted from the light emitting element passes.

The second light shielding film may include a light reflecting material.

In a case where the second light shielding film includes the light reflecting material, the first light shielding film may include a light absorbing material.

The biological information measuring device may be provided with a third light shielding film provided on a side surface of the lid.

The light emitting element may be a light emitting element that emits coherent light.

The biological information measuring device may be provided with a housing that accommodates the light emitting element and the light receiving element in the housing, in which it is possible that a partition wall is not present between the light emitting element and the light receiving element in the housing.

The biological information measuring device may be a blood flow measuring device.

### BRIEF DESCRIPTION OF DRAWINGS

FIG. 1 is a schematic plan view illustrating a part of a biological information measuring device 1.

FIG. 2 is a cross-sectional view taken along line A-A of the biological information measuring device 1 illustrated in FIG. 1.

FIG. 3 is a schematic plan view illustrating a configuration example of a first light shielding film 10.

FIG. 4 is a schematic plan view illustrating a part of a biological information measuring device 1A.

FIG. 5 is a cross-sectional view taken along line B-B of the biological information measuring device 1A illustrated in FIG. 4.

FIG. 6 is a cross-sectional view of a biological information measuring device 1B.

### MODE FOR CARRYING OUT THE INVENTION

Hereinafter, a preferred mode for carrying out the present technology is described with reference to the drawings. Note that embodiments hereinafter described are representative embodiments of the present technology, and the scope of the present technology is not narrowed by them. The description is given in the following order.

1. First embodiment (example provided with first light shielding film)

2. Second embodiment (example provided with second light shielding film)

3. Third embodiment (example provided with third light shielding film)

### 1. First Embodiment

First, a biological information measuring device 1 according to a first embodiment of the present technology is described.

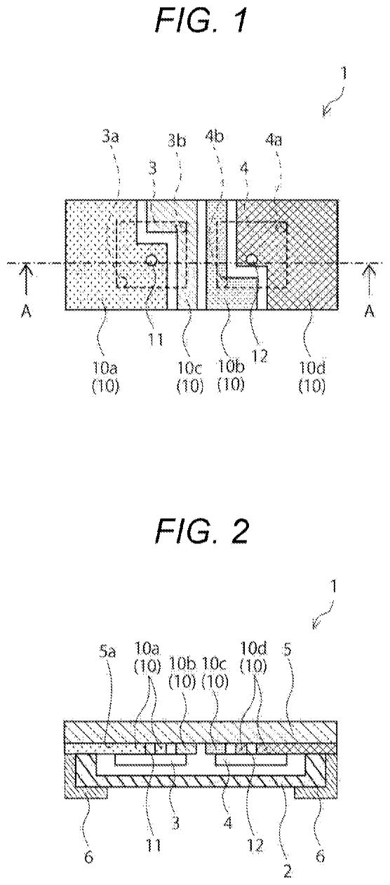

Examples of biological information that may be measured by using the biological information measuring device of the present technology include, for example, a blood flow volume, a blood cell volume, a blood flow speed, a pulse rate and the like. Among them, the biological information measuring device of the present technology is suitable as a blood flow measuring device for measuring information regarding the blood flow, and is particularly preferably used as laser Doppler flowmetry. Hereinafter, the laser Doppler flowmetry as an example of the biological information measuring device of the present technology is described with reference to FIGS. 1 to 3.

FIG. 1 is a schematic plan view illustrating a part of the biological information measuring device 1. FIG. 2 is a cross-sectional view taken along line A-A of the biological information measuring device 1 illustrated in FIG. 1. In FIG. 1, a lid 5 illustrated in FIG. 2 is not illustrated.

The biological information measuring device 1 is provided with a housing 2, a light emitting element 3, and a light receiving element 4. The housing 2 has an inner cavity with top open. The light emitting element 3 and the light receiving element 4 are accommodated in the housing 2. As the light emitting element 3, a light emitting element that emits coherent light is suitable, and examples thereof include a laser diode and the like, for example. Examples of the light receiving element 4 include, for example, a photodiode, a phototransistor and the like. Although FIGS. 1 and 2 illustrate a configuration in which one light emitting element 3 and one light receiving element 4 are provided, the number of light emitting elements 3 and that of light receiving elements 4 are not limited to one, and may be two or larger. The number of light emitting elements 3 and that of light receiving elements 4 may be the same as or different from each other.

The biological information measuring device 1 is further provided with a plate-shaped lid 5 having translucency. The lid 5 is arranged so as to cover an opening at the top of the housing 2, and faces a bottom surface of the housing 2. It is sufficient that a material forming the lid 5 may transmit at least light emitted from the light emitting element 3, and it is possible that light other than the light emitted from the light emitting element 3 is not transmitted. For example, in a case where infrared light is used as a light source of the light emitting element 3, the lid 5 may include a material that transmits only the infrared light and absorbs light other than the infrared light. From the viewpoint of preventing incidence of unnecessary light on the light receiving element 4, the lid 5 preferably includes a material that does not transmit the light other than the light emitted from the light emitting element 3. Examples of the material forming the lid 5 include glass, resin and the like, for example.

The lid 5 is provided with a first light shielding film 10 having conductivity on a first principal surface 5a. The first principal surface 5a of the lid 5 is a surface directed to the housing 2 side and faces the bottom surface of the housing 2. The light emitting element 3 and the light receiving element 4 are electrically joined to the first light shielding film 10 on the first principal surface 5a. When a positional relationship among the lid 5, the first light shielding film 10, the light emitting element 3, and the light receiving element 4 is checked with reference to the cross-sectional view illustrated in FIG. 2, the first light shielding film 10 is arranged below the lid 5, and the light emitting element 3 and the light receiving element 4 are arranged below the first light shielding film 10. When joining the light emitting element 3 and the light receiving element 4 to the first light shielding film 10, flip chip mounting is preferable.

The first light shielding film 10 includes a first light emitting side opening 11 through which the light emitted from the light emitting element 3 passes, and a first light receiving side opening 12 through which light received by the light receiving element 4 passes. The first light emitting side opening 11 is provided in a position corresponding to the light emission center of the light emitting element 3 so that the light emitted from the light emitting element 3 may pass therethrough. The first light receiving side opening 12 is provided in a position corresponding to the light receiving surface center of the light receiving element 4 so that the light received by the light receiving element 4 may pass therethrough.

In this manner, in the biological information measuring device 1 of the present technology, the light emitting element 3 and the light receiving element 4 are directly joined to the first light shielding film 10, so that positioning between the light emitting element 3 and the first light emitting side opening 11 and positioning between the light receiving element 4 and the first light receiving side opening 12 may be performed with high accuracy. As a result, it is possible to suppress positional displacement between the light emitting element 3 and the first light emitting side opening 11 and between the light receiving element 4 and the first light receiving side opening 12.

A lower limit of a diameter of the first light emitting side opening 11 is preferably equal to or larger than a diameter of the light emitting center of the light emitting element 3. An upper limit of the diameter of the first light emitting side opening 11 is preferably equal to or smaller than the diameter of the light emitting center of the light emitting element 3+100 μm, and more preferably equal to or smaller than the diameter of the light emitting center of the light emitting element 3+10 μm. A lower limit of a diameter of the first light receiving side opening 12 is preferably equal to or larger than a diameter of the light receiving surface center of the light receiving element 4. An upper limit of the diameter of the first light receiving side opening 12 is preferably equal to or smaller than the diameter of the light receiving surface center of the light receiving element 4+100 μm, and more preferably equal to or smaller than the diameter of the light receiving surface center of the light receiving element 4+10 μm.

The first light shielding film 10 described above is preferably divided into three or more sections independent from each other so that terminals of the light emitting element 3 and the light receiving element 4 may be individually driven. The “sections independent from each other” mean the sections configured to be electrically separated, and in other words, they are the “sections electrically separated from each other”. It is preferable that at least one terminal of the light emitting element 3 or the light receiving element 4 is electrically joined to each section of the first light shielding film 10.

As an example, FIGS. 1 and 2 illustrate a configuration in which the first light shielding film 10 is divided into four sections 10a, 10b, 10c, and 10d, and an anode terminal 3a of the light emitting element 3, a cathode terminal 3b of the light emitting element 3, an anode terminal 4a of the light receiving element 4, and a cathode terminal 4b of the light receiving element 4 are electrically joined to the respective sections. Specifically, the anode terminal 3a of the light emitting element 3 is joined to the section 10a, the cathode terminal 3b of the light emitting element 3 is joined to the section 10b, the cathode terminal 4b of the light receiving element 4 is joined to the section 10c, and the anode terminal 4a of the light receiving element 4 is joined to the section 10d.

Each of the sections 10a, 10b, 10c, and 10d of the first light shielding film 10 is individually connected to connection wiring and electrically connected to a circuit board (not illustrated) through the connection wiring. FIG. 2 illustrates members for connection wiring 6 and 6 located lateral to the housing 2, and the members for connection wiring 6 and 6 are in contact with a circuit board (not illustrated) located below.

As described above, in the example illustrated in FIGS. 1 and 2, the first light shielding film 10 is divided into the sections independent from each other, and the terminals 3a and 3b of the light emitting element 3 and the terminals 4a and 4b of the light receiving element 4 are electrically joined to different sections, respectively. Furthermore, the connection wiring is individually connected to each section of the first light shielding film 10. With such a configuration, the terminals 3a and 3b of the light emitting element 3 and the terminals 4a and 4b of the light receiving element 4 may be independently electrically driven.

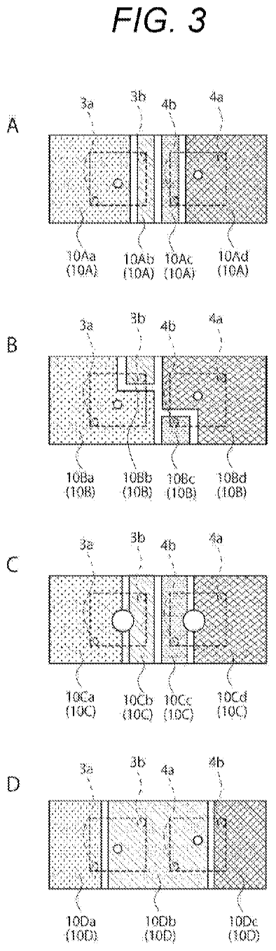

FIG. 3 illustrates a schematic plan view illustrating a configuration example of the first light shielding film 10. A shape of each section of the first light shielding film 10 is not particularly limited, and may be, for example, a shape illustrated in FIG. 3. Each of first light shielding films 10A to 10C illustrated in FIGS. 3A to C, respectively, is divided into four independent sections, and the terminals 3a and 3b of the light emitting element 3 and the terminals 4a and 4b of the light receiving element 4 are electrically joined to different sections, respectively.

Unlike in FIGS. 3A to C, a first light shielding film 10D illustrated in FIG. 3D is divided into three independent sections 10Da, 10Db, and 10Dc. In an example illustrated in FIG. 3D, the anode terminal 3a of the light emitting element 3 is joined to the section 10Da, the cathode terminal 3b of the light emitting element 3 and the anode terminal 4a of the light receiving element 4 are joined to the section 10Db, and the cathode terminal 4b of the light receiving element 4 is joined to the section 10Dc. In a case where the cathode terminal 3b of the light emitting element 3 and the anode terminal 4a of the light receiving element 4 are connected to the ground, as illustrated in FIG. 3D, a plurality of terminals connected to the ground may be joined to the same section.

A material forming the first light shielding film 10 may be any material that blocks light, and may be a light reflecting material or a light absorbing material. From the viewpoint of improving usage efficiency of light in the measurement of the biological information, the first light shielding film 10 is preferably a light reflecting film including a light reflecting material.

Here, an operation of the biological information measuring device 1 is described. When using the biological information measuring device 1, in a state in which the lid 5 is in contact with user's skin, light is emitted from the light emitting element 3 toward the skin. The emitted light passes through the first light emitting side opening 11 of the first light shielding film 10, the lid 5, and the skin surface and reaches the blood vessel under the skin. The light scattered by the blood cells in the blood flowing through the blood vessel passes through the lid 5 and the first light receiving side opening 12 of the first light shielding film 10 to be detected by the light receiving element 4. The biological information measuring device 1 obtains the biological information such as the blood flow speed by analyzing the detected scattered light.

By using the light reflecting film as the first light shielding film 10 as described above, it is possible to return light that does not pass through the first light receiving side opening 12 out of the light that passes through the user's skin and backscattered toward the light receiving element 4 side to the user's skin side again by the first light shielding film 10. In this manner, it is possible to improve the usage efficiency of light by returning the scattered light that cannot be received by the light receiving element 4 to the skin side to reuse.

The first light shielding film 10 is preferably formed by metal vapor deposition. As a metal used for the metal vapor deposition, a metal having high adhesion to the lid 5 is suitable. For example, in a case where the material forming the lid 5 is glass, the first light shielding film 10 is preferably a nickel-deposited film.

According to the biological information measuring device 1 of the present technology, the light emitting element 3 and the first light emitting side opening 11 may be positioned with high accuracy as described above, so that it is possible to suppress the positional displacement between the light emitting element 3 and the first light emitting side opening 11. By the way, in general, in a case where relative positions of a light emitting element and an opening corresponding to the light emitting element formed on a light shielding film are displaced, if a diameter of the opening is small, a part of light is blocked and lost. Therefore, the diameter of the opening corresponding to the light emitting element needs to be formed large in consideration of the positional displacement. On the other hand, if the diameter of the opening corresponding to the light emitting element is too large, an original function of the light shielding film to block stray light and electrical noise is deteriorated, which is not desirable. On the other hand, in the biological information measuring device 1 of the present technology, since it is possible to precisely adjust relative positions of the light emitting element 3 and the first light emitting side opening 11, it is not necessary to make the diameter of the first light emitting side opening 11 large in consideration of the positional displacement as described above. That is, by using the present technology, the diameter of the first light emitting side opening 11 may be made smaller than that of the conventional technology. As a result, the biological information measuring device 1 of the present technology may effectively block stray light and electrical noise to improve measurement accuracy of the biological information.

Furthermore, in general, if relative positions of a light receiving element and an opening corresponding to the light receiving element formed on a light shielding film are displaced, an amount of light that reaches the light receiving element decreases, so that it is necessary to increase a size of the light receiving element in consideration of the positional displacement. On the other hand, as the size of the light receiving element increases, an outer shape of a biological information measuring device also becomes large accordingly. On the other hand, in the biological information measuring device 1 of the present technology, since it is possible to precisely adjust relative positions of the light receiving element 4 and the first light receiving side opening 12, it is not necessary to increase the size of the first light receiving side opening 12 in consideration of the positional displacement as described above. That is, according to the present technology, the light receiving element having a small outer shape may be used, and as a result, an outer shape of an entire device may be made compact.

Furthermore, in the conventional technology, it is necessary to design a space in a housing wide in consideration of positional displacement when attaching the light emitting element and the light receiving element in the housing, so that the outer shape of the entire device becomes large. However, in the biological information measuring device 1 of the present technology, the light emitting element 3 and the light receiving element 4 are joined to the first light shielding film 10, and it is not necessary to take into consideration attaching tolerance of the light emitting element 3 and the light receiving element 4 in the design of the housing 2, so that the outer shape of the entire device may be made compact.

A problem of the increase in size of the entire device is particularly remarkable in a case where a partition wall is arranged between the light emitting element and the light receiving element as in the technology disclosed in Patent Document 2 described above. The partition wall between the light emitting element and the light receiving element is provided in order to prevent generation of optical noise due to direct incidence of the light emitted from the light emitting element on the light receiving element. In order to provide the partition wall, it is necessary to secure a certain level of space in the housing, and as a result, the entire device becomes large. On the other hand, in the biological information measuring device 1 of the present technology, the light emitting element 3 and the light receiving element 4 are joined to the first light shielding film 10, and all the light emitted from the light emitting element 3 passes through the first light emitting side opening 11 and the lid 5, so that the light is not directly incident on the light receiving element 4 from the light emitting element 3. That is, in the biological information measuring device 1 of the present technology, it is not necessary to provide a partition wall that prevents direct incidence of the light emitted from the light emitting element 3 on the light receiving element 4 between the light emitting element 3 and the light receiving element 4 in the housing 2. Therefore, according to the present technology, the outer shape of the entire device may be made smaller than that of the conventional technology.

Moreover, in a case of manufacturing the housing provided with the partition wall as in the conventional technology, a manufacturing method is limited as compared with that of a housing not provided with a partition wall, so that it is difficult to reduce a manufacturing cost of the housing. However, according to the present technology, it is not necessary to provide the partition wall in the housing 2, and a degree of freedom in a manufacturing method is high, so that it is possible to reduce a cost.

In this embodiment, the laser Doppler flowmetry (LDF) has been described as an example of the biological information measuring device. The laser Doppler flowmetry may irradiate the human skin surface with a laser beam to measure a blood flow in the capillary non-invasively and continuously, and this is small in size. Therefore, the biological information measuring device 1 of this embodiment is suitable for a wearable type blood flow meter such as a headband type, a neckband type, and a belt type. Other embodiments of the biological information measuring device according to the present technology include, for example, any personal digital assistant (PDA) such as a smartphone and a tablet terminal; any electronic device such as a medical device, a game device, or a home appliance and the like.

Furthermore, the present technology is also suitable for the biological information measuring device that measures pulse using photoplethysmography (PPG) and the biological information measuring device that measures pulse and blood flow by combining PPG and LDF technologies.

### 2. Second Embodiment

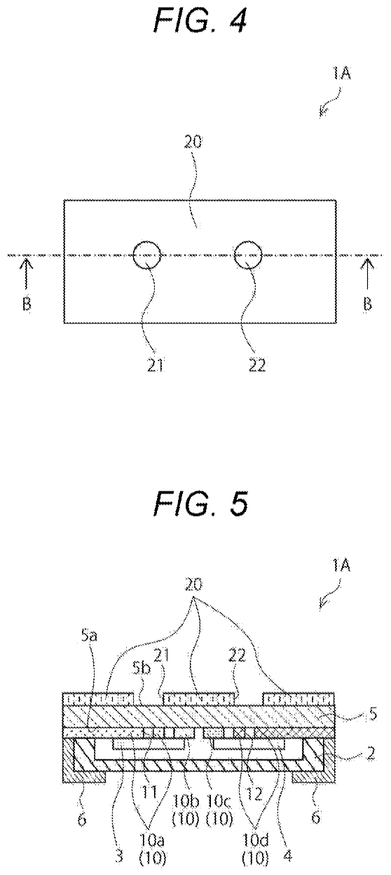

Next, a biological information measuring device 1A according to a second embodiment of the present technology is described with reference to FIGS. 4 and 5. In FIG. 5, components similar to those illustrated in FIG. 2 are assigned with the same reference numerals, and the description thereof is omitted as appropriate.

FIG. 4 is a schematic plan view illustrating a part of the biological information measuring device 1A. FIG. 5 is a cross-sectional view taken along line B-B of the biological information measuring device 1A illustrated in FIG. 4. The biological information measuring device 1A of the second embodiment is provided with a second light shielding film 20 in addition to the configuration of the first embodiment.

The second light shielding film 20 is provided on a second principal surface 5b facing a first principal surface 5a of a lid 5. The second light shielding film 20 includes a second light emitting side opening 21 through which light emitted from a light emitting element 3 passes, and a second light receiving side opening 22 through which light received by a light receiving element 4 passes. The second light emitting side opening 21 is provided in a position corresponding to the light emission center of the light emitting element 3 so that the light emitted from the light emitting element 3 may pass therethrough. The second light receiving side opening 22 is provided in a position corresponding to the light receiving surface center of the light receiving element 4 so that the light received by the light receiving element 4 may pass therethrough.

Advantages of this embodiment are hereinafter described. LDF being a suitable example of the present technology is a blood flow meter that utilizes an interference phenomenon between light reflected without hitting the blood out of light backscattered from the human body and light that hits the blood so that a wavelength is slightly Doppler-shifted. The light backscattered from the human body exhibits a pattern referred to as speckle. The LDF detects a speed at which this speckle pattern changes, and a light receiving element observes a value obtained by integrating the speckle pattern over an entire light receiving surface. At that time, if the speckle pattern is small with respect to a light receiving area of the light receiving element, the speckle pattern is averaged, so that the change in speckle pattern is homogenized and measurement becomes difficult, and it is possible that a change in signal by a blood flow cannot be captured. Therefore, it is preferable to make the number of speckle pattern generated in the light receiving element (average number of speckle pattern=average area of speckle pattern/area of light receiving surface of light receiving element) as small as possible. One of means for decreasing the number of speckle pattern is to decrease the diameter of the first light receiving side opening 12 described in the first embodiment. As another means, it is possible to limit the light that reaches the light receiving element 4 and increase the average area of the speckle pattern by providing a further opening in a position separated from the light receiving element 4. The opening located in the position separated from the light receiving element 4 corresponds to the second light receiving side opening 22 described in the second embodiment.

The average area of the speckle pattern in a case where the second light receiving side opening 22 is provided is specifically described. In a case where the first light receiving side opening 12 formed on the first light shielding film 10 is provided, an average diameter of the speckle pattern on the light receiving surface of the light receiving element 4 is about a wavelength of light. Therefore, in order to prevent the homogenization of the speckle pattern, it is necessary to decrease the diameter of the first light receiving side opening 12. On the other hand, when the diameter of the first light receiving side opening 12 is made too small, an amount of light that passes through the first light receiving side opening 12 decreases, and it is possible that a sufficient S/N ratio cannot be secured.

In a case where the second light receiving side opening 22 formed on the second light shielding film 20 is provided, an average area P_{coh} of the speckle pattern on the light receiving surface of the light receiving element 4 is expressed by following equation (1), where A represents a wavelength and Ω represents a solid angle subtended by the second light receiving side opening 22 as seen from the light receiving element 4.

A_{coh}=λ²/Ω (1)

In this manner, by providing the second light receiving side opening 22 in the position separated from the light receiving element 4, Ω becomes smaller and the average area of the speckle pattern increases, and as a result, the average number of speckle pattern on the light receiving element 4 decreases. That is, by providing the second light receiving side opening 22, the change in speckle pattern caused by movement of the blood is not homogenized, and it is possible to measure biological information with a better S/N ratio.

A material forming the second light shielding film 20 may be any material that blocks light, and may be a light reflecting material or a light absorbing material. As in the first embodiment, from the viewpoint of improving usage efficiency of light in the measurement of the biological information, the second light shielding film 20 is preferably a light reflecting film including a light reflecting material. In a case where the second light shielding film 20 includes the light reflecting material, the first light shielding film 10 may be a light absorbing film including a light absorbing material. By using the light absorbing film as the first light shielding film 10, it is possible to reduce unnecessary stray light that does not pass through the human body. Furthermore, the second light shielding film 20 may have conductivity as is the case with the first light shielding film 10. In this case, the second light shielding film is preferably grounded.

In the biological information measuring device 1A illustrated in FIGS. 4 and 5, the second light shielding film 20 is formed so as to cover the portion corresponding to the light emitting element 3, and the second light shielding film 20 is provided with the second light emitting side opening 21 in the portion corresponding to the light emitting element 3. However, the second light emitting side opening 21 is not an essential component, and it is also possible that the second light shielding film 20 is not formed so as to cover the portion corresponding to the light emitting element 3. In a case where the light absorbing film is used as the first light shielding film 10, it is preferable from the viewpoint of improving the usage efficiency of light that the second light shielding film 20 is also provided in the position corresponding to the light emitting element 3 and includes the second light emitting side opening 21.

### 3. Third Embodiment

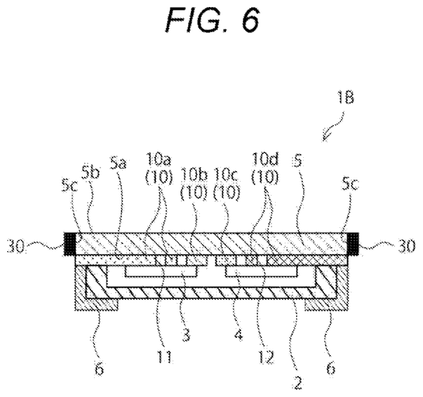

Next, a biological information measuring device 1B according to a third embodiment of the present technology is described with reference to FIG. 6. In FIG. 6, components similar to those illustrated in FIG. 2 are assigned with the same reference numerals, and the description thereof is omitted as appropriate.

FIG. 6 is a cross-sectional view of the biological information measuring device 1B. The biological information measuring device 1B of the third embodiment is provided with third light shielding films 30 and 30 provided on side surfaces 5c and 5c of a lid 5 in addition to the configuration of the first embodiment. By providing the third light shielding film 30, it is possible to prevent light incident on the side surface 5c of the lid 5 from propagating in the lid 5 to be incident on the light receiving element 4 and become optical noise.

The side surfaces 5c and 5c of the lid 5 are surfaces orthogonal to a first principal surface 5a and a second principal surface 5b. The biological information measuring device 1B illustrated in FIG. 6 is provided with two third light shielding films 30, but the number of third light shielding films 30 is not particularly limited and may be one or larger.

The third light shielding film 30 is not particularly limited as long as this is a film having a light shielding property, but this preferably is a film formed by black printing.

It is possible to combine two or more feature parts out of the feature parts related to the present technology described above. That is, the various feature parts described in each embodiment may be arbitrarily combined irrespective of the embodiments. For example, the second light shielding film 20 according to the second embodiment may be combined with the third embodiment.

Note that the present technology may also have following configurations.

[1] A biological information measuring device, provided with:

a plate-shaped lid having translucency; a first light shielding film provided on a first principal surface of the lid and having conductivity; a light emitting element; and a light receiving element,

in which the light emitting element and the light receiving element are electrically joined to the first light shielding film, and

the first light shielding film includes a first light emitting side opening through which light emitted from the light emitting element passes, and a first light receiving side opening through which light received by the light receiving element passes.

[2] The biological information measuring device according to [1],

in which the first light shielding film is divided into three or more sections independent from each other, and

at least one of the terminal of the light emitting element or the terminal of the light receiving element is respectively joined to the sections.

[3] The biological information measuring device according to [1] or [2], in which the first light shielding film includes a light reflecting material.
[4] The biological information measuring device according to any one of [1] to [3], further provided with:

a second light shielding film provided on a second principal surface facing the first principal surface of the lid,

in which the second light shielding film includes a second light receiving side opening through which light received by the light receiving element passes.

[5] The biological information measuring device according to [4], in which the second light shielding film includes a second light emitting side opening through which light emitted from the light emitting element passes.
[6] The biological information measuring device according to [4] or [5], in which the second light shielding film includes a light reflecting material.
[7] The biological information measuring device according to [6], in which the first light shielding film includes a light absorbing material.
[8] The biological information measuring device according to any one of [1] to [7], further provided with: a third light shielding film provided on a side surface of the lid.
[9] The biological information measuring device according to any one of [1] to [8], in which the light emitting element emits coherent light.
[10] The biological information measuring device according to any one of [1] to [9], further provided with:

a housing that accommodates the light emitting element and the light receiving element in the housing,

in which a partition wall is not present between the light emitting element and the light receiving element in the housing.

[11] The biological information measuring device according to any one of [1] to [10], being a blood flow measuring device.

### REFERENCE SIGNS LIST

1, 1A, 1B Biological information measuring device
2 Housing
3 Light emitting element
3a, 3b Terminal of light emitting element
4 Light receiving element
4a, 4b Terminal of light receiving element
5 Lid
5a First principal surface of lid
5b Second principal surface of lid
5c Side surface of lid
6 Member for connection wiring
10 First light shielding film
11 First light emitting side opening
12 First light receiving side opening
20 Second light shielding film
21 Second light emitting side opening
22 Second light receiving side opening
30 Third light shielding film

What is claimed is:

1. A biological information measuring device comprising:
a plate-shaped lid having translucency; a first light shielding film provided on a first principal surface of the lid and having conductivity; a light emitting element; and a light receiving element,
wherein the light emitting element and the light receiving element are electrically joined to the first light shielding film, and
the first light shielding film includes a first light emitting side opening through which light emitted from the light emitting element passes, and a first light receiving side opening through which light received by the light receiving element passes.

2. The biological information measuring device according to claim 1,
wherein the first light shielding film is divided into three or more sections independent from each other, and
at least one of the terminal of the light emitting element or the terminal of the light receiving element is respectively joined to the sections.

3. The biological information measuring device according to claim 1,
wherein the first light shielding film includes a light reflecting material.

4. The biological information measuring device according to claim 1, further comprising:
a second light shielding film provided on a second principal surface facing the first principal surface of the lid,
wherein the second light shielding film includes a second light receiving side opening through which light received by the light receiving element passes.

5. The biological information measuring device according to claim 4, wherein the second light shielding film includes a second light emitting side opening through which light emitted from the light emitting element passes.

6. The biological information measuring device according to claim 4, wherein the second light shielding film includes a light reflecting material.

7. The biological information measuring device according to claim 6, wherein the first light shielding film includes a light absorbing material.

8. The biological information measuring device according to claim 1, further comprising: a third light shielding film provided on a side surface of the lid.

9. The biological information measuring device according to claim 1, wherein the light emitting element emits coherent light.

10. The biological information measuring device according to claim 1, further comprising:
a housing that accommodates the light emitting element and the light receiving element in the housing,
wherein a partition wall is not present between the light emitting element and the light receiving element in the housing.

11. The biological information measuring device according to claim 1, being a blood flow measuring device.
